(19) United States
(12) Patent Application Publication
(10) Pub. No.: US 2007/0088428 A1
(43) Pub. Date: Apr. 19, 2007

(54) Intraluminal device with asymmetric cap portion

(75) Inventor: Eyal Teichman, Hod-Hasharon (IL)

Correspondence Address: RISSMAN JOBSE HENDRICKS & OLIVERIO, LLP, ONE STATE STREET, SUITE 800, BOSTON, MA, 02109, US

(73) Assignee: Cappella, Inc., Auburndale, MA (US)

(21) Appl. No.: 11/532,362

(22) Filed: Sep. 15, 2006

Related U.S. Application Data
(60) Provisional application No. 60/717,303, filed on Sep. 15, 2005.

(51) Int. Cl. A61F 2/06

(52) U.S. Cl. 623/1.16; 623/1.35

(57) ABSTRACT

A device is provided for placement at a bifurcation of a vessel. The device comprises an anchor portion having a proximal end, a distal end, and an anchor body connecting the proximal and distal ends. The anchor body comprises a series of struts configured to provide a radial force to a wall of the vessel. A cap portion is positioned proximal to the anchor portion. A plurality of protruding elements for extension into an ostial region of the vessel are provided on the cap portion and at least one of the protruding elements is longer than at least another one of the protruding elements.

### RELATED APPLICATIONS

This application is a non-provisional application of, and claims priority to, provisional patent application Ser. No. 60/717,303 filed Sep. 15, 2005 and entitled “Intraluminal Device With Asymmetric Cap Portion,” the entire subject matter and contents of which are incorporated herein by reference for all purposes.

### FIELD OF THE INVENTION

The invention relates to intraluminal devices for treatment at ostial regions of a vessel.

### BACKGROUND OF THE INVENTION

In today's society, many people suffer from a buildup of a plaque layer covering one or more segments of a coronary vessel where the lesion obstructs the flow of blood through the vessel. This buildup is referred to as a coronary lesion. Often, this condition is treated by placing medical devices or appliances within a patient for supporting the blood vessels or other lumens within the body that have been re-enlarged following cardio balloon angioplasty.

With regard to angioplasty, typically an endovascular or intraluminal implant known as a stent is placed within the blood vessel. A stent is usually tubular in shape and may have a lattice or connected-wire tubular construction. The stent is usually placed within the vessel in a compressed state and then allowed to expand. The support structure of the stent is designed to prevent early collapse of a vessel that has been weakened and damaged by angioplasty. The support provided by the stent prevents the vessel from either closing, referred to as restenosis, or from suffering spasms shortly after the angioplasty procedure. The support has been shown to facilitate the healing of the damaged vessel wall, a process that occurs over a number of months. Self-expanding and balloon-expandable stents are well known.

During the healing process, it is thought that inflammation caused by angioplasty and stent implant injury causes smooth muscle cell proliferation and regrowth inside the stent. This cell proliferation and regrowth closes the flow channel, i.e., restenosis, thereby reducing or eliminating the beneficial effect of the angioplasty/stenting procedure. Blood clots may also form inside of the newly implanted stent due to the thrombotic nature of the stent surfaces, even when biocompatible materials are used to form the stent.

While large blood clots may not form during the angioplasty procedure itself, or immediately after the procedure, due to the current practice of injecting powerful anti-platelet drugs into the blood circulation, some thrombosis is always present, at least on a microscopic level on stent surfaces. This microscopic thrombosis is thought to play a significant role in the early stages of restenosis by establishing a biocompatible matrix on the surfaces of the stent whereupon smooth muscle cells may subsequently attach and multiply.

There are stent coatings that contain bioactive agents designed to reduce or eliminate thrombosis or restenosis. Such bioactive agents may be dispersed or dissolved in either a bio-durable or bio-erodible polymer matrix that is attached to the surface of the stent wires prior to implant. After implantation, the bioactive agent diffuses out of the polymer matrix and into the surrounding tissue over a period lasting at least four weeks, and in some cases up to one year or longer. Ideally, the duration of diffusion is chosen to match the time course of restenosis, smooth muscle cell proliferation, thrombosis or a combination thereof.

Some coronary lesions may develop in coronary bifurcations, i.e., a bifurcated vessel including a main vessel associated via an ostial region with a side-branch vessel. Bifurcation lesions may be categorized according to the location of the lesion in the bifurcated vessel. In one example, a type 4a bifurcation lesion may refer to a lesion on the wall of the main vessel in proximity to the ostial region.

Treating bifurcation lesions, e.g., type 4a lesions, using the conventional methods described above, may result in at least part of the plaque layer “drifting” into the side-branch. This effect, commonly referred to as “the snow-plow effect,” may lead to a partial blockage of the side-branch, which may be treated by deploying one or more additional stents into the bifurcated vessel.

Conventional methods for treating bifurcation lesions may include deploying a first stent part in the main vessel covering the side branch, and then inflating a “kissing balloon” and deploying a second stent part in the side branch, thereby to form a “T-stent” structure. Such methods as these, however, may result in the T-stent disrupting/obstructing the blood flow from the main vessel to the side branch.

Other stenting methods and/or specially designed bifurcation stents, for example, the Jostent® B stent, the Invatec Bifurcation stent, or the AST stent, may be relatively bulky and may have limited tractability, limited maneuverability and limited access to small caliber vessels. Moreover, other stenting methods do not provide adequate protection at varying angles of bifurcation.

### SUMMARY OF THE INVENTION

In one embodiment, a device for positioning at a bifurcation of a vessel comprises: an anchor portion having a proximal end, a distal end, and an anchor body connecting said proximal and distal ends, said anchor body comprising a series of struts configured to provide a radial force to a wall of the vessel; and a cap portion positioned proximal to said anchor portion, said cap portion comprised of multiple protruding elements for extension into an ostial region of said vessel, wherein at least one of said multiple protruding elements is longer than at least another one of said multiple protruding elements.

Adjacent protruding elements may be of different lengths from one another. Alternatively, at least one pair of adjacent protruding elements comprises protruding elements with different lengths from one another.

In one embodiment, the anchor body is substantially cylindrical with a substantially constant diameter along its length. Alternatively, the anchor body is cylindrical with a diameter that linearly increases from the distal end to the proximal end. Still further, the anchor body may be cylindrical and flare at the proximal end.

In yet another embodiment, the multiple protruding elements are circumferentially positioned about a proximal opening of the cap portion; and a shortest protruding element is at a position on the circumference substantially opposite a largest protruding element.

A device for positioning at a bifurcation of a vessel comprising a substantially cylindrical anchor portion having a proximal end and a distal end; a cap portion having a proximal end and a distal end coupled to the proximal end of the anchor portion; and a plurality of protruding elements circumferentially disposed about a proximal opening at the proximal end of the cap portion, wherein at least one protruding element is longer than at least one other protruding element is provided.

In one embodiment, a shortest protruding element is at a position on the circumference substantially opposite a largest protruding element.

### BRIEF DESCRIPTION OF THE DRAWINGS

The above and further advantages of the invention may be better understood by referring to the following description in conjunction with the accompanying drawings in which:

FIGS. 1A and 1B are schematic illustrations of bifurcated vessels including main vessels and side branch vessels;

FIGS. 1C and 1D are schematic illustrations of the bifurcated vessels of FIGS. 1A and 1B, with an intraluminal device positioned in side branches of the bifurcated vessels;

FIG. 2 is a perspective illustration of an intraluminal device in accordance with exemplary embodiments of the invention;

FIG. 3 is an illustration of a flattened view of the intraluminal device of FIG. 2, showing the geometric configuration and patterns in accordance with exemplary embodiments of the present invention;

FIG. 4 is a perspective illustration of an intraluminal device including connectors, in accordance with exemplary embodiments of the invention;

FIG. 5 is an illustration of a flattened view of the intraluminal device of FIG. 4, showing the geometric configuration and patterns in accordance with exemplary embodiments of the present invention;

FIG. 6 is a perspective illustration of an intraluminal device including an articulating module, in accordance with exemplary embodiments of the invention;

FIG. 7 is an illustration of a flattened view of the intraluminal device of FIG. 6, showing the geometric configuration and patterns in accordance with exemplary embodiments of the present invention;

FIGS. 8A and 8B are schematic illustrations showing an intraluminal device positioned in bifurcated vessels having a first angle of bifurcation and a second angle of bifurcation, respectively; and

FIGS. 9A-9C are schematic illustrations showing varying shapes of an intraluminal device in accordance with embodiments of the present invention.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the invention. It will be understood by those of ordinary skill in the art that the present invention may be practiced without these specific details. In other instances, well-known methods, procedures, components and structures may not have been described in detail so as not to obscure the present invention.

It is to be understood that the present invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is further appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Embodiments of the invention may include an intraluminal device configured to selectively protect at least part of a predetermined region, e.g., an ostial region, of a bifurcated vessel and/or to dispense medication substantially uniformly across at least part of the predetermined region, as described below.

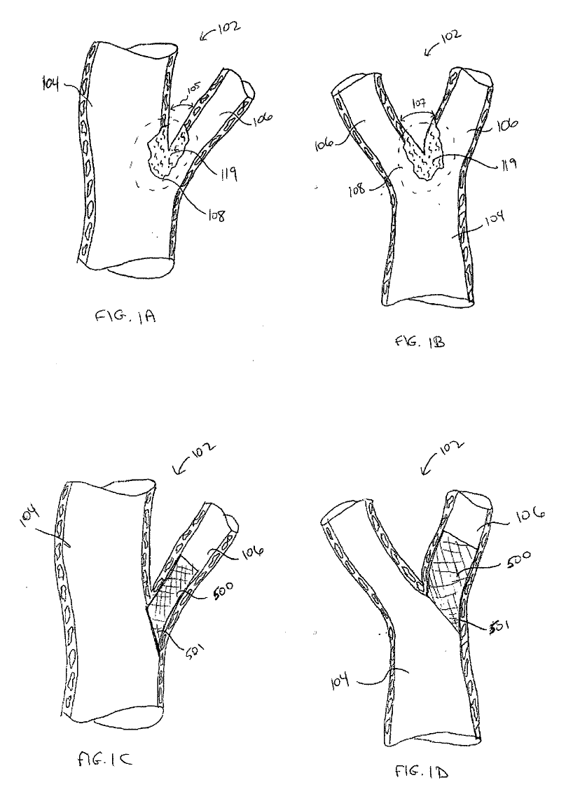

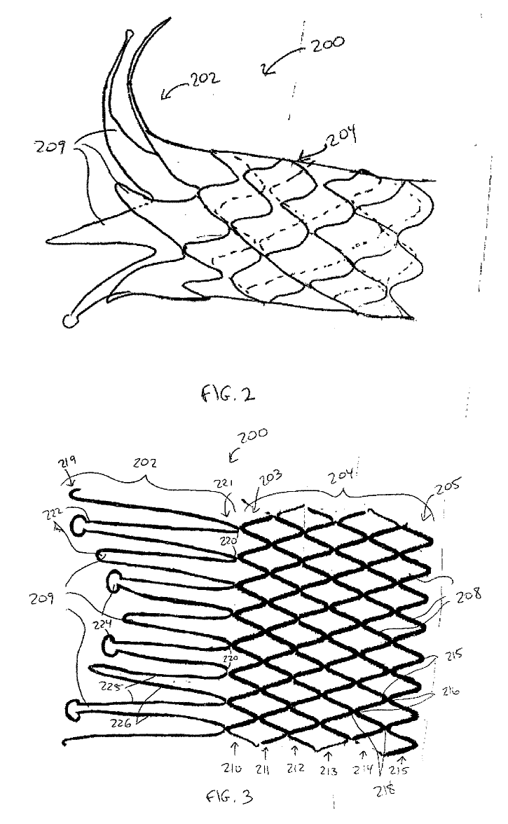

Reference is now made to FIGS. 1A and 1B, which are schematic illustrations of bifurcated vessels. FIG. 1A depicts a bifurcated vessel 102 including a main vessel 104 and a side branch vessel 106 extending from main vessel 104. An angle of bifurcation 105 is defined as the angle between main vessel 104 and side branch vessel 106. Embodiments of the present invention are particularly useful for relatively small angles of bifurcation, such as angles ranging from 10 to 60 degrees. FIG. 1B depicts a bifurcated vessel 102 having a main vessel 104 and two side branch vessels or arms 106, extending from main vessel 104. Such bifurcations are commonly known as “Y” bifurcations. A “Y” angle of bifurcation 107 is defined as the angle between the two side branch vessels 106. Embodiments of the present invention are particularly useful for relatively small angles of bifurcation, such as angles ranging from 0 to 60 degrees. Bifurcated vessel 102 may include a target tissue, for example, a diseased segment (a “lesion”), that may include a plaque layer 119 obstructing the flow of blood through the diseased segment of the vessel. The lesion may be located along at least part of main vessel 104, side branch vessel 106 and/or an ostial region 108 between side-branch vessel 106 and main vessel 104.

Reference is now made to FIGS. 1C and 1D, which are schematic illustrations of the bifurcated vessels of FIGS. 1A and 1B, with an intraluminal device 200 positioned in side branches 106 of bifurcated vessels 102. Intraluminal device 500 has an asymmetric proximal end 501, for optimal protection of side branch 106. In one embodiment, a main stent is further positionable in main vessel 104. In another embodiment, intraluminal device 500 is a stand-alone device. Devices of the present invention are configured to provide protection to an ostial region of a vessel, while avoiding excess deformation of the vessel.

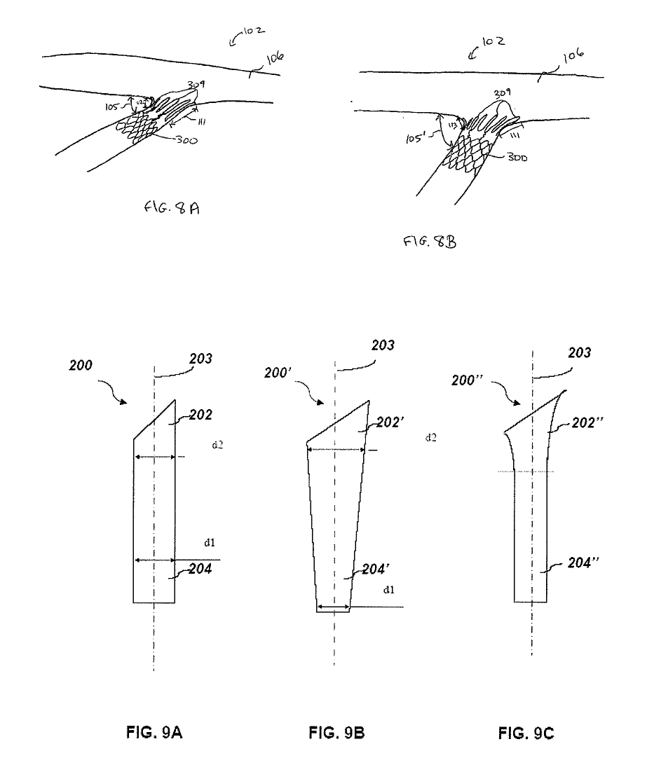

Reference is now made to FIGS. 9A-9C, which are schematic illustrations showing shapes of intraluminal device 200 in accordance with various embodiments of the present invention. Intraluminal device 200 is illustrated with respect to a central axis 203, and includes an anchor portion 204 and a cap portion 202. In one embodiment, shown in FIG. 9A with respect to intraluminal device 200, anchor portion 204 has a diameter d1 which is relatively constant along central axis 203. In another embodiment, shown in FIG. 9B with respect to intraluminal device 200′, anchor portion 204′ has a first diameter d1 at a distal end thereof and a second diameter d2 at a proximal end thereof, with respect to central axis 203. More specifically, the diameter of anchor portion 204′ may increase in a proximal direction, so as to form a substantially conical shape. In yet another embodiment, shown in FIG. 9C with respect to intraluminal device 200″, cap portion 202″ is configured in a trumpet-like shape, wherein a section of cap portion 202″ which is adjacent to anchor portion 204″ is curved or shaped outwardly with respect to central axis 203. The embodiment shown in FIG. 9C may include a substantially constant diameter along anchor portion 204″, as in intraluminal device 200 shown in FIG. 9A, or may include a variable diameter along anchor portion 204″, as in intraluminal device 200′ shown in FIG. 9B.

Reference is now made to FIG. 2, which is a perspective illustration of an intraluminal device 200, in accordance with exemplary embodiments of the invention. Intraluminal device 200 includes an anchor portion 204 and a cap portion 202.

According to exemplary embodiments of the invention, anchor portion 204 may have a generally tubular, e.g., spring-like, structure, which may be circularly symmetrical with respect to a central axis. In other embodiments, anchor portion 204 has a geometric configuration of struts, as described in detail below. In some embodiments, anchor portion 204 has a generally conical structure, wherein a distal portion thereof has a smaller diameter than a proximal portion thereof. Anchor portion 204 is configured to hold intraluminal device 200 in place in the vessel, preventing shifting of the device. An outer diameter of anchor portion 204 may be compatible with, i.e., approximately equal to or slightly larger than, an inner diameter of the side branch vessel 106. According to some exemplary embodiments of the invention, the outer diameter of anchor portion 204 may be substantially constant along a central axis. According to other embodiments, the outer diameter of anchor portion 204 may vary along a central axis, e.g., in order to enable an improved positioning and/or “anchoring” of the anchor portion 204 with respect to the side branch 106 and/or to ease the insertion of the intraluminal device 200 into the side branch. For example, anchor portion 204 may have a generally conical shape, i.e., the outer diameter of anchor portion 204 may monotonically, i.e., linearly, increase or decrease along the central axis.

According to exemplary embodiments of the invention, cap portion 202 includes multiple protruding elements 209 extending in a proximal direction. In exemplary embodiments, multiple protruding elements 209 are configured to extend into, or in a direction toward, ostial region 108. The number of multiple protruding elements 209 is chosen based on the particular anatomy in which intraluminal device 200 is to be placed. Furthermore, the lengths of each of multiple protruding elements 209 may vary, thus providing an asymmetrical cap portion 202. For example, the lengths of multiple protruding elements 209 may vary so as to form an angled edge of intraluminal device 200. For example, longest multiple protruding elements 209 may be in a range of 4-10 mm in length, while shortest multiple protruding elements may be in a range of 1-5 mm in length. These configurations allow for better protection of ostial region 108 at bifurcations of various angles. Upon deployment of intraluminal device 200, multiple protruding elements 209 extend outwardly, forming a trumpet shape, and protecting areas of ostial region 108 that are frequently not adequately protected due to the configurations of known intraluminal devices. In some embodiments, a diameter of a proximal portion of intraluminal device 200 is in a range of 1-3 times larger than a diameter of a distal portion of intraluminal device 200.

Reference is now made to FIG. 3, which is an illustration of intraluminal device 200 in a flattened view, showing the geometric configuration and patterns in accordance with exemplary embodiments of the present invention. Anchor portion 204 has an anchor portion proximal end 203 and an anchor portion distal end 205, wherein anchor portion proximal end 203 is at least partially connected to other portions of intraluminal device 200 as described hereinbelow. Anchor portion 204 is comprised of struts or supporting elements 208, which are interconnected to provide support to an inner portion of the side branch vessel 106. In some embodiments, supporting elements 208 form a uniform or repeating cell pattern, such as repeating diamond shapes, hexagonal shapes, or any other pattern. In alternative embodiments, supporting elements 208 form non-uniform patterns, having variations in pattern dimensions and/or strut characteristics. In one embodiment, supporting elements 208 are configured in a series of interconnected columns, for example, columns 210-215 shown in FIG. 3. It should be readily apparent that the number of columns may vary, and that the number of columns shown and described herein with respect to the present embodiment is for illustrative purposes only. Each column 210-215 has a sinusoidal pattern having peaks 215 and valleys 216, wherein peaks 215 are defined as elements protruding in a direction facing anchor portion distal end 205 and valleys 216 are defined as elements protruding in a direction facing anchor portion proximal end 203. Adjacent columns are 180 degrees out of phase in their sinusoidal patterns, such that a peak 215 of one column, for example column 210, is in line with a valley 216 of an adjacent column, for example column 211. This configuration can be repeatedly applied to additional columns, such that any desired number of columns may be included. Columns 210-215 are connected to one another at contact areas 218 between peaks 215 of one column and valleys 216 of an adjacent column. In alternative embodiments, adjacent columns are in phase with one another, or out of phase by other degrees. A length of anchor portion 304 may be in a range of 4-40 mm when in an unexpanded state, and may have a diameter in a range of 2-6 mm in a fully expanded state.

Cap portion 202 includes multiple protruding elements 209 configured, for example, in a sinusoidal pattern having cap peaks 220 and cap valleys 222, wherein cap peaks 220 are defined as elements facing a distal side 221 of cap portion 202 and cap valleys 222 are defined as elements facing a proximal side 219 of cap portion 202. Cap peaks 220 and cap valleys 222 are connected by upper segments 225 and lower segments 226 that are repeatedly angled in one direction and in the opposite direction, such that upper segments 225 are connected to lower segments 226 alternatingly at proximal side 219 forming cap valleys 222 and at a distal side 221 forming cap peaks 220. In alternative embodiments, protruding elements 209 are comprised of other patterns, including non-angled upper and lower segments, rounded, squared or any other suitable configuration. In exemplary embodiments, multiple protruding elements 209 are longer than supporting elements 208 of individual columns of anchor portion 204, and are configured to extend into or in a direction of ostial region 108. Some of protruding elements 209 further include tip portions 224 at their proximal ends. In one embodiment, only some of protruding elements 209 (such as every alternate one, for example) include a tip portion 224. In other embodiments, every protruding element 309 includes a tip portion 224. Tip portions 224 provide additional surface area for delivery of medication, and are also suitable for placing of markers, e.g., radio-opaque, thereon. In some embodiments, multiple protruding elements 209 are in a range of 1-6 mm in length. After shaping, a diameter defined by cap peaks 220 may be in a range of 3-10 mm. More particularly, longest multiple protruding elements 209 may be in a range of 4-10 mm in length, while shortest multiple protruding elements may be in a range of 1-5 mm in length.

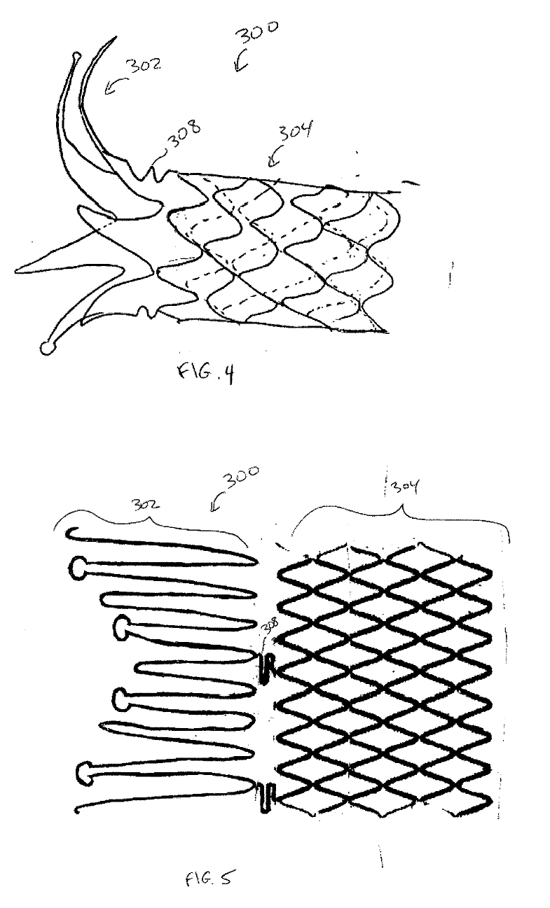

Reference is now made to FIG. 4 and FIG. 5, which are a perspective illustration and a flattened view, respectively, of an intraluminal device 300, in accordance with exemplary embodiments of the invention. Intraluminal device 300 includes an anchor portion 304 and a cap portion 302, wherein anchor portion 304 and cap portion 302 are connected by connectors 308. Connectors 308 may be curved, straight, S shaped, or any other suitable configuration. The presence of cap connectors 308 provides flexibility to intraluminal device, and allows for some amount of rotational and axial shift while being positioned in a vessel.

Reference is now made to FIG. 6 which is a perspective illustration of an intraluminal device 400 in accordance with exemplary embodiments of the invention. Intraluminal device 400 includes an anchor portion 404, a cap portion 402, and an articulating module 406. Articulating module 406 includes cap connectors 432 connecting a body 430 of articulating module 406 to cap portion 402, and anchor connectors 434 connecting body 430 to anchor portion 404. In some embodiments, cap connectors 432 include two connectors, separated from each other by 180 degrees around body 430, and anchor connectors 434 include two connectors, separated from each other by 180 degrees around body 430, and further positioned at approximately 90 degrees from cap connectors 432 around body 430. Thus, cap connectors 432 may be flexed back and forth in one direction or plane and anchor connectors 434 may be flexed back and forth in another direction or plane which is orthogonal to the direction of flexing of cap connectors 432, providing multiple directional flexibility overall by articulating module 406. In some embodiments, flexing of cap connectors 432 and anchor connectors 434 is variable, such that either one or both of cap connectors 432 and anchor connectors 434 can be flexed in multiple directions. In some embodiments, cap connectors 432 and anchor connectors 434 are pre-shaped for specific angles, requiring less force for flexing at the specific angles. In some embodiments, only one cap connector 432 and/or one anchor connector 434 is used. Body 430 can be of various designs and geometries, but should be designed such that it can be crimped to a smaller diameter and expanded upon deployment of intraluminal device 400. Examples of such designs are described more fully hereinbelow.

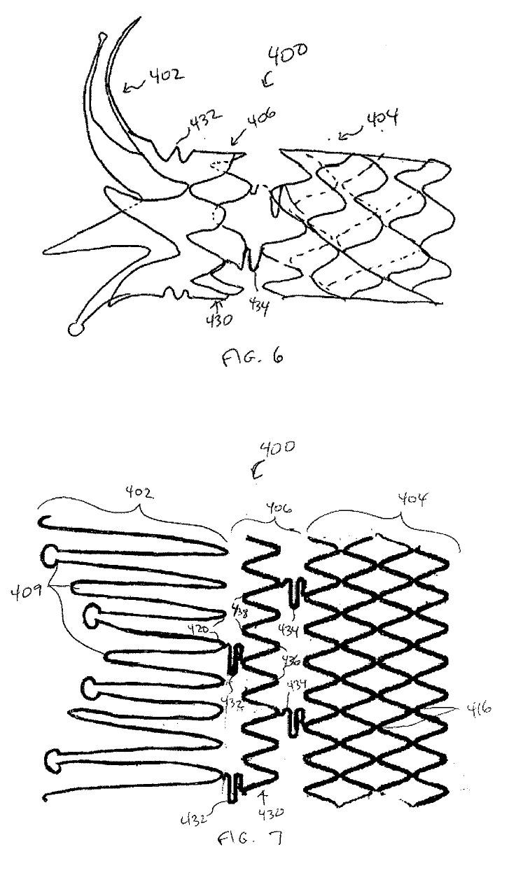

Reference is now made to FIG. 7, which is a flattened view of intraluminal device 400 in accordance with exemplary embodiments of the invention. Cap portion 402 and anchor portion 404 are designed in accordance with cap portions and anchor portions described in earlier embodiments. Articulating module 406 is provided between anchor portion 404 and cap portion 402, and includes a body 430, cap connectors 432 and anchor connectors 434. A purpose of articulating module 406 is to provide a small radius of curvature between anchor portion 404 and cap portion 402, so that intraluminal device 400 can bend at many different angles without significant additional rotation. A further purpose of articulating module 406 is to provide a small spring-like mechanism for correction of axial positioning of cap portion 402 within a vessel. Body 430 may have a similar geometric pattern or configuration as anchor portion 404, or may have a different pattern or configuration. A length of body 430 is minimized so as to ensure maximum flexing capabilities. For example, a length of body 430 may be in a range of 0.5-4 mm. In one embodiment, body 430 includes a row of interconnecting struts having a sinusoidal pattern having peaks 436 and valleys 438, wherein peaks 436 are defined as elements protruding in a direction facing anchor portion 404 and valleys 438 are defined as elements protruding in a direction facing cap portion 402, as shown in FIG. 7. In another embodiment, body 430 includes several rows of interconnecting struts. Rows of interconnecting struts may be configured in identical or in varying patterns, and may be connected to one another by body connectors. In the embodiment shown in FIG. 7, anchor connectors 434 are disposed between peaks 436 of articulating module 406 and valleys 416 of anchor portion 404. Furthermore, cap connectors 432 are disposed between valleys 438 of articulating module 406 and peaks 420 of cap portion 402. In exemplary embodiments, anchor connectors 434 are spaced apart from one another so as to provide a high degree of flexibility between articulating module 406 and anchor portion 404, and cap connectors 432 are spaced apart from one another so as to provide a high degree of flexibility between articulating module 406 and cap portion 402. For example, anchor connectors 434 may be placed on one of every five or six peaks 436 of articulating module 406, and cap connectors 436 may be placed on one of every five or six valleys 438 of articulating module 406, such that anchor connectors 434 and cap connectors 436 are alternatingly positioned along body 430. In some embodiments, the struts of body 430 of articulating module 406 are shorter than at least some of the struts of protruding elements 409 of cap portion 402. In some embodiments, anchor connectors 334 and cap connectors 436 are straight connectors. In other embodiments, anchor connectors 434 and cap connectors 436 are curved connectors, spiral connectors, or S-shaped connectors, as shown in FIG. 7. In some embodiments, anchor connectors 434 and cap connectors 436 are pre-shaped. In some embodiments, anchor connectors 434 do not have the same configuration as cap connectors 436. It should be readily apparent that different numbers of connectors as well as different configurations of struts, connectors, and protruding elements and patterns related thereto may vary, and that all such possibilities are within the scope of the present invention.

The intraluminal devices of the present invention may be configured to protect the ostial region 108 and/or the side branch vessel 106 by selectively covering at least part of an inner wall of the ostial region 108 in order, for example, to prevent the plaque layer 119 or parts thereof from migrating into the side branch vessel 106 by the snow-plow effect, which may result from applying the angioplasty device.

According to exemplary embodiments of the invention, the intraluminal devices of the present invention may be formed of a generally elastic, super-elastic, in-vivo stable and/or “shape-memorizing” material, i.e., a material able to be initially formed in a desired shape, e.g., during an initial procedure performed at relatively high temperature, to be deformed, e.g., compressed, and to assume the desired shape in which it was previously shaped. Intraluminal devices of the present invention may be formed of Nickel-Titanium alloy (“nitinol”) wire that possesses both super-elastic and shape-memorizing properties. The wire may have a diameter of between 30 and 300 micrometers. In other embodiments, biocompatible non-elastic materials, such as stainless steel, for example, may be used.

In some embodiments, the intraluminal device is formed from a wire. In other embodiments, the intraluminal device is cut from a single tube. The intraluminal device may be formed from a single piece of material or may be assembled in sections. In an alternative embodiment, cap portions may be of a different material than anchor portions. Cap portions may be formed from any compliant material known to one of ordinary skill in the art, e.g., a polymeric material. Further, cap portions may be formed from a non-compliant material.

According to exemplary embodiments of the invention, at least part of the intraluminal device may be coated with a layer of a desired medication or a material having desired properties to carry and subsequently apply and/or dispense a desired medication. Anchor portions and/or cap portions may be coated with a controlled-release polymer and/or drug, as known in the art, for reducing the probability of undesired side effects, e.g., restenosis. Restenosis may occur as a result of a percutaneous procedure performed on the bifurcated vessel 102, e.g., including insertion of an angioplasty device into the bifurcated vessel 102.

In some embodiments, anchor portion is configured to provide support to the vessel, while the cap portion is configured to deliver medication to the ostial region. In other embodiments, the cap portion is configured to deliver medication and to provide support in conjunction with the anchor portion. Accordingly, the radial forces of the intraluminal device may be substantially constant along the length of the device, or may be variable along the length of the device.

Reference is now made to FIG. 8A, which is a schematic illustration showing intraluminal device 300 positioned in a bifurcated vessel 102 having a first angle of bifurcation 105, for example 30 degrees, and to FIG. 8B, which is a schematic illustration showing intraluminal device 300 positioned in a bifurcated vessel having a second angle of bifurcation 105′, for example 60 degrees. As shown, the longest of multiple protruding elements 309 is configured to cover a long section 111 of the wall of the ostium, while the shortest of multiple protruding elements 309 is configured to cover a short section 113 of the wall of the ostium. As the angle of bifurcation increases, the longest of multiple protruding elements 309 protrudes further into main vessel 104. Alternatively, lengths of multiple protruding elements 309 can be designed for particular angles of bifurcation or ranges of angles of bifurcation. Thus, for example, a device configured for use with a 10-45 degree angle of bifurcation might have a first set of multiple protruding elements 309 having lengths in a range of 1-5 mm for the shortest of multiple protruding elements 309 and lengths in a range of 4-10 mm for the longest of multiple protruding elements 309. A device configured for use with a 30-60 degree angle of bifurcation might have a second set of multiple protruding elements 309 having lengths in a range of 1-5 mm for the shortest of multiple protruding elements 309 and lengths in a range of 3-8 mm for the longest of multiple protruding elements 309. It should be readily apparent that the lengths of multiple protruding elements 309 may by any suitable length for covering both sides of a wall of an ostium. It should further be apparent that any of the intraluminal devices described herein, or any other configuration of intraluminal devices having an anchor portion and a cap portion may have similar protruding elements with varying lengths.

Although some embodiments of the invention described above may refer to an intraluminal device configured for capping a bifurcated coronary vessel and for dispensing medication, it will be appreciated by those skilled in the art that the intraluminal device according to other embodiments of the invention may be configured for capping any other bifurcated lumen, artery or vessel, e.g., in the vascular, biliary, genitourinary, gastrointestinal and respiratory systems, which may have narrowed, weakened, distorted, or otherwise deformed, and/or for dispensing any other substance across at least part of the lumen, artery or vessel, e.g., the carotid artery or trachea bifurcations.

The medicinal coating can include, e.g., and not meant to be limiting, any one or more of the following: paclitaxel, rapamycin, and heparin.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. A device for positioning at a bifurcation of a vessel, the device comprising:
an anchor portion having a proximal end, a distal end, and an anchor body connecting said proximal and distal ends, said anchor body comprising a series of struts configured to provide a radial force to a wall of the vessel; and
a cap portion positioned proximal to said anchor portion, said cap portion comprised of multiple protruding elements for extension into an ostial region of said vessel, wherein at least one of said multiple protruding elements is longer than at least another one of said multiple protruding elements.

2. The device of claim 1, wherein adjacent protruding elements are of different lengths from one another.

3. The device of claim 1, wherein at least one pair of adjacent protruding elements comprises protruding elements with different lengths from one another.

4. The device of claim 1, wherein at least one of the multiple protruding elements comprises a radio-opaque marker.

5. The device of claim 1, wherein the anchor body is substantially cylindrical with a substantially constant diameter along its length.

6. The device of claim 1, wherein the anchor body is cylindrical with a diameter that linearly increases from the distal end to the proximal end.

7. The device of claim 1, wherein the anchor body is cylindrical and flares at the proximal end.

8. The device of claim 1, wherein:
the multiple protruding elements are circumferentially positioned about a proximal opening of the cap portion; and
a shortest protruding element is at a position on the circumference substantially opposite a largest protruding element.

9. A device for positioning at a bifurcation of a vessel, the device comprising:
a substantially cylindrical anchor portion having a proximal end and a distal end;
a cap portion having a proximal end and a distal end coupled to the proximal end of the anchor portion; and
a plurality of protruding elements circumferentially disposed about a proximal opening at the proximal end of the cap portion,
wherein at least one protruding element is longer than at least one other protruding element.

10. The device of claim 8, wherein the anchor body is substantially cylindrical with a substantially constant diameter along its length.

11. The device of claim 8, wherein the anchor body is cylindrical with a diameter that linearly increases from the distal end to the proximal end.

12. The device of claim 8, wherein the anchor body is cylindrical and flares at the proximal end.

13. The device of claim 8, wherein:
a shortest protruding element is at a position on the circumference substantially opposite a largest protruding element.
